(19) United States
(12) Patent Application Publication
(10) Pub. No.: US 2024/0148987 A1
(43) Pub. Date: May 9, 2024

(54) DEVICE FOR DELIVERY OF AEROSOLIZED DRUG IN A PORTION OF A BODY

(71) Applicant: CUROTHERM TECHNO SOLUTIONS LLP, Mumbai (IN)

(72) Inventors: Nirav Mehta, Mumbai (IN); Palkesh Agrawal, Indore (IN)

(21) Appl. No.: 18/411,786

(22) Filed: Jan. 12, 2024

Related U.S. Application Data
(63) Continuation of application No. PCT/IN2022/050633, filed on Jul. 12, 2022.

(30) Foreign Application Priority Data
Jul. 12, 2021 (IN) 202121031193

(51) Int. Cl. A61M 11/00; H04R 17/00

(52) U.S. Cl. CPC A61M 11/005; H04R 17/00; A61M 2202/04; A61M 2210/1021

(57) ABSTRACT

A device, for delivery of aerosolized drug in a portion of a body, comprising: a nozzle (100) comprising a head portion extending, distally, into an elongate shaft (108), said head portion comprising: piezoelectric transducers (104, 106) mounted between electrically conductive electrode discs (103, 105), said piezoelectric transducers (104, 106) creating capillary waves in a liquid film causing atomization of drug/s while passing through said nozzle (100); an aperture (101) on said nozzle (100), through which tubing (112) is connected, said aperture (101) follows through with a passage (300) ensconced in said elongate shaft (108), and passing through said piezoelectric transducers (104, 106) and said electrically conductive electrode discs (103, 105) in order to allow passage of drug received through said tubing (112); and said elongate shaft (108) supporting a body member (107) at its operative proximal end and having an opening (109), said drug being dispensed through said opening (109).

### RELATED APPLICATIONS

This application claims priority under 35 U.S.C. § 120 to, and is a continuation of, co-pending International Application PCT/IN2022/050633, filed Jul. 12, 2022 and designating the US, which claims priority to IN Application 202121031193, filed Jul. 12, 2021, such IN Application also being claimed priority to under 35 U.S.C. § 119. These IN and International applications are incorporated by reference herein in their entireties.

### FIELD

This invention relates to the field of biomedical engineering.

Particularly, this invention relates to a device for delivery of aerosolized drug in a portion of a body.

### BACKGROUND

Chemotherapy is the use of drugs to destroy cancer cells. It usually works by keeping the cancer cells from growing, dividing, and making more cells. Because cancer cells usually grow and divide faster than normal cells, chemotherapy has more of an effect on cancer cells. In chemotherapy, many drugs require injection directly into a vein; this is called intravenous (IV) treatment.

In comparison to intravenous (IV) treatment, intraperitoneal (IP) administration results in a several-fold increase in drug concentration within an abdominal cavity.

There is now growing evidence from clinical studies showing a survival advantage for IP chemotherapy in various tumor types, including ovarian, thoracic, gastric, and colorectal cancer. Efficacy of intraperitoneal (IP) chemotherapy is limited by poor distribution within the abdominal cavity and by poor tissue penetration.

Therefore, there is a need for a new way for administering intraperitoneal chemotherapy into an abdominal cavity.

Prior art technology which is termed as Pressurized Intraperitoneal Aerosol Chemotherapy (PIPAC) administers chemotherapy drug through laparoscopic access using two balloon trocars in an operating room equipped with laminar air flow. In a first step, a normothermic capno-peritoneum is established with a pressure of 12 mmHg. In a second step, a cytotoxic solution (about 10% of a normal systemic dose) is aerosolized with a pressure injector into an abdominal cavity and maintained for 30 minutes. In a third step, the aerosol is then removed through a closed suction system.

### SUMMARY

However, there is a need to move away from the prior art technology of delivery of aerosol under pressure. Prior art mechanisms have to rely on third party pressure for delivery and hence are inefficient. These third-party pressure delivery mechanisms are injectors which create pressure for drug feeding into a nozzle of the prior art. These pressure injectors are bulky equipment, typically, available in bigger sized hospitals and predominantly used as a radiolucent die injection in patient for CTs, MRIs, and CATHLAB departments.

This restricts such treatment in smaller size hospitals which doesn't have Pressure injectors.

There is a need for a device, mechanism, apparatus, and/or system which does not rely on pressure and/or any pressure injector and/or any third-party equipment for delivery.

An object of the invention is to provide a new way for administering intraperitoneal chemotherapy into an abdominal cavity.

Another object of the invention is to provide a device in order to deliver chemotherapy drug into an abdomen of a palliative patient in an aerosolized form and the drug delivery being of average nanometer size or smaller.

Yet another object of the invention is to provide a device in order to deliver chemotherapy drug, in an aerosol form, under ultrasound waves.

Yet another object of the invention is to provide an easier delivery mechanism for delivery of chemotherapy drug into an abdomen of a palliative patient.

Yet another object of the invention is to provide a safe operability of the device ensuring safety for OT/OR personnel present, while delivering the aerosolized chemotherapy drug into an abdomen.

Yet another object of the invention is to provide possibility of safe maneuverability of the drug delivery nozzle while delivering aerosolized chemotherapy drug for better reach within the peritoneal cavities.

Still another object of the invention is to provide a device, which does not rely on pressure and/or any pressure injector and/or any third-party equipment; for delivery.

An additional object of the invention is to provide a device which is convenient to use and also at small sized hospitals.

According to this invention, there is provided a device for delivery of aerosolized drug in a portion of a body, said device comprising:
a nozzle comprising a head portion extending, distally, into an elongate shaft, said head portion comprising:
   one or more piezoelectric transducers mounted between a pair of electrically conductive electrode discs, said piezoelectric transducers configured to create capillary waves in a liquid film leading to atomization of drug/s while passing through said nozzle, said piezoelectric transducers having pre-stress ranging between 1.8 N/sq mm to 3.25 N/sq mm in order to attain optimized amplitude of standing waves;
   an aperture, at an operative proximal end of said nozzle, through which tubing is connected, said aperture follows through with a passage ensconced in said elongate shaft, and passing through said piezoelectric transducers and said electrically conductive electrode discs in order to allow passage of drug received through said tubing; and
said elongate shaft supporting a body member at its operative proximal end and having an opening at its operative distal end, said drug, received through said tubing, and passing through said passage, being dispensed through said opening.

In at least an embodiment, ratio, of cross-sectional area of body member to elongated shaft, being in the range of 11 to 14, in order to attain maximum amplification factor for a given length of said nozzle.

In at least an embodiment, said piezoelectric transducers are disc-shaped piezoelectric transducers.

In at least an embodiment, said body member is a cylindrical body member with its flat operative top surface being configured to support said nozzle.

In at least an embodiment, fastening mechanisms is provided at operative top of said nozzle to hold said piezoelectric transducers from its operative top.

In at least an embodiment, body member is provided at operative bottom of said nozzle to hold said piezoelectric transducers from its operative bottom.

In at least an embodiment, said tubing is connected to said nozzle, through said aperture, by means of a luer connector.

In at least an embodiment, said tubing is a gravity-fed tubing.

In at least an embodiment, a cable applies electric potential to said piezoelectric transducers.

In at least an embodiment, said cylindrical body member extends away from said discs to form an elongate shaft having a diameter substantially lesser than the diameter of the cylindrical body member.

In at least an embodiment, a conical surface is provided adjacent to said opening, said conical surface tapers such that it is narrowest adjacent to said opening of said elongate shaft.

In at least an embodiment, a step is provided at an operative distal end of said elongate shaft adjacent to said opening.

In at least an embodiment, a controller is provided to control frequency of vibration of said piezoelectric transducer which, in turn, atomizes drug from its liquid form into a nano-particle mist before being dispensed through said opening.

In at least an embodiment, working frequency of said device is in the range of 20-80 KHz.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be described in relation to the accompanying drawings, in which:

FIG. 1 is an isometric view of a nozzle of the device of this invention;

FIG. 2 is a top elevation view of the nozzle illustrated in FIG. 1;

FIG. 3 is a cross-sectional view of the nozzle constructed in accordance with the present invention showing the principal elements thereof;

FIG. 4 illustrates a block diagram for the working of the device of this invention;

FIG. 5 illustrates that smaller particle size ranging from 46 nm to 176 nm sizes enhances the absorption of drug in the tissue; and

FIG. 6 illustrates increased depth of drug penetration up to 800 μm in the tissue due to smaller drug particle size.

### DETAILED DESCRIPTION

According to this invention, there is provided a device for delivery of aerosolized drug in a portion of a body.

This device, of this invention, facilitates a new way in the application of administering intraperitoneal (IP) chemotherapy in the form of aerosol, typically, into the abdominal cavity.

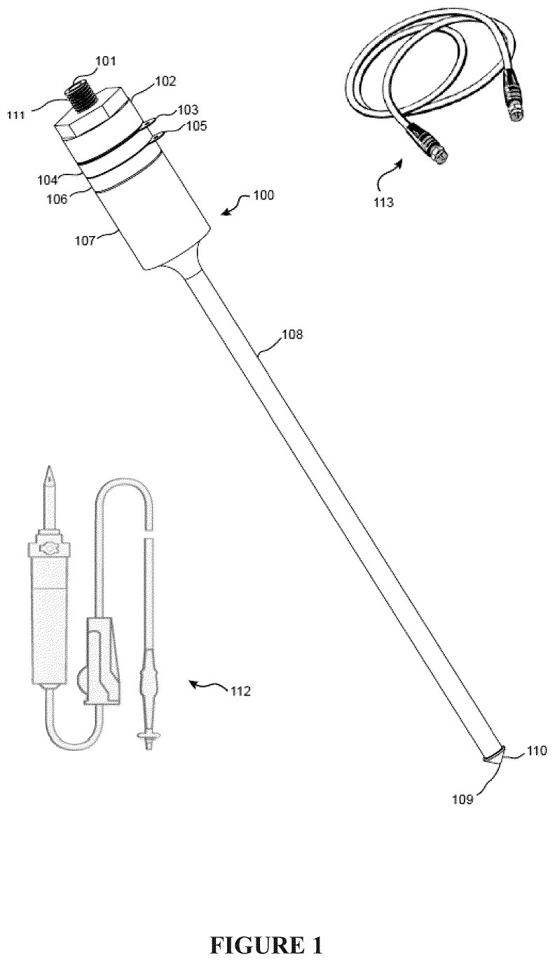

FIG. 1 is an isometric view of the nozzle.

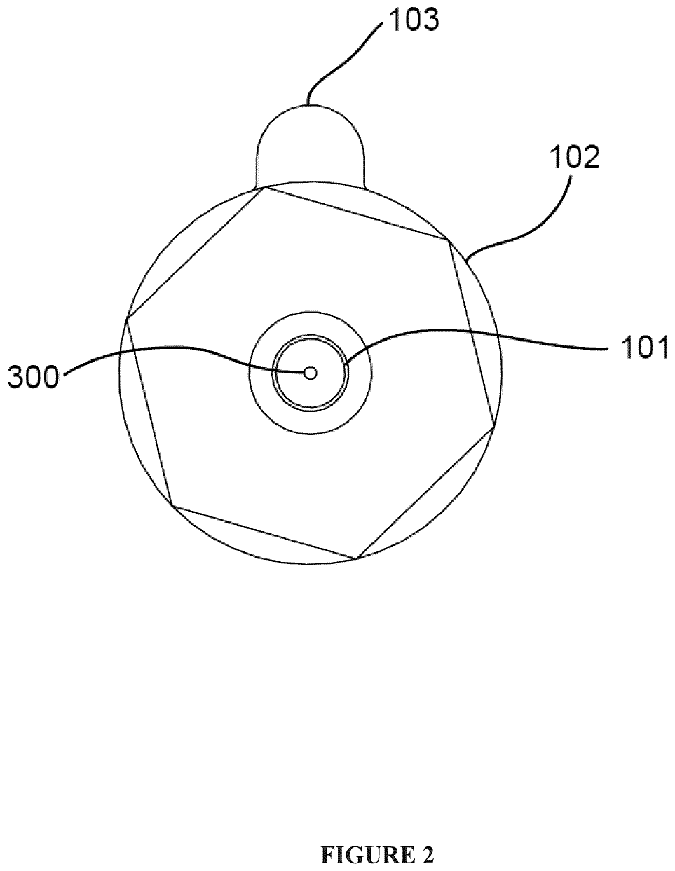

FIG. 2 is a top elevation view of the nozzle illustrated in FIG. 1.

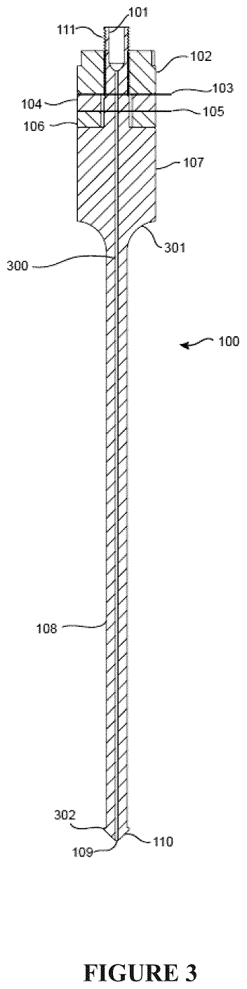

FIG. 3 is a cross-sectional view of the nozzle constructed in accordance with the present invention showing the principal elements thereof.

In at least an embodiment, the device comprises a nozzle (100). The nozzle (100) comprises one or more piezoelectric transducers (104, 106) that creates capillary waves in a liquid film leading to atomization of drug/s while passing through the nozzle (100). Typically, the nozzle (100) comprises one or more disc-shaped piezoelectric transducers (104, 106) mounted between a pair of electrically conductive electrode discs (103, 105). An electric potential is applied through a RF coaxial cable (113).

Referring to the drawings, and in particular to FIG. 1, the nozzle 100, in accordance, with this invention is illustrated. Nozzle (100), which forms a head portion, comprises a disc shaped piezoelectric transducer (104, 106) mounted between a pair of electrically conductive electrode discs (103, 105) and a flat surface of a cylindrical body member (107) (being a support member). An electric potential is applied through RF coaxial cable (113). Cylindrical body members (107) and fastening mechanisms (102) are holding bodies (top and bottom) of piezoelectric transducer (104, 106). There is an aperture (101) on a rear body member (111) of the nozzle (100) through which a tube (112) (which may be an IV set) is connected with the help of luer connector. Aperture (101) has a passage (300) through which drug flows. The cylindrical body member (107) extends away from the discs (103, 105) to form an elongate shaft (108) having a diameter substantially lesser than the diameter of the cylindrical body member (107). An opening (109) is provided at an operative distal end of the nozzle (100) i.e., at the free end of the elongate shaft (108) (nozzle stem); through which drug is dispensed. A conical surface (110) is provided adjacent to the opening (109); the conical surface (110) tapers such that it is narrowest adjacent to the opening (109) of the elongate shaft (108). For flow rate and nozzle co-relation, the nozzle's conical surface (110) is sandblasted to lower cohesive force between liquid atoms by making the surface rough which results in fine atomization at even higher flow rate.

Referring to FIG. 3, passage (300) within the elongate shaft (108) allows for drug to flow before being dispensed through the opening (109). Reference numeral 301 refers to a curvature of a nozzle (100) to accommodate for change in diameter from cylindrical body member (107) to its connected elongate shaft (108). At the distal end, a step (302) is provided.

In at least an embodiment, the device comprises a controller which can be used to control frequency of vibration of the piezoelectric transducer (104) which, in turn, atomizes the diluted drug from its liquid form into a nano-particle mist which can be then sprayed/delivered into an abdominal cavity through the nozzle (100) of this invention. The nozzle is customized or tailor made to be compatible to the controller and specific to the stated application

The size of particles generated by the device, of this invention, depends on the frequency of vibration, surface tension, and viscosity of the liquid. The particle size is inversely proportional to frequency and, thus, the frequency can be increased to reduce the particle size. In this device, liquid is not forced through a small orifice but flows through a large channel in which ultrasonic waves produce the aerosol.

In at least an embodiment, the nozzle (100) is connected to the controller unit through the coaxial RF cable (113); through which an electric potential is applied. A tubing (110) is connected through a luer connector connected on the nozzle (100). Cylindrical body members (107) and fastening mechanism (102) depict a holding body of the piezoelectric transducer (104, 106). There is an aperture (101) on the rear body member (111) of the nozzle (100) through which the tubing (112) is connected with the help of luer connector.

The nozzle stem (108) comprises a conical surface (110), at its operative distal end, such that the nozzle stem's (108) narrowest part is adjacent to the opening (109). Reference numeral 300 refers to a passage for drug flow.

In at least an embodiment, actuation by the controller, it starts identification of the nozzle (100). This phase checks the status of the nozzle i.e., being used already or a fresh nozzle (100). Upon again acknowledging the identification phase and pressing the key, the controller starts delivering power to the nozzle (100). A pinch valve, which is present in the tubing (112), is to be opened, and drug starts atomizing from the tip of the nozzle (100).

Typically, this device's working frequency is in the range of 20-80 KHz.

Two piezoelectric transducers are housed between body members (107 and 102) with a pre-stress ranging between 1.8 N/sq mm and 3.25 N/sq mm to attain optimized amplitude of standing waves. The ratio of cross-sectional area of body member (107) with that of elongated shaft (108) is in the region of 11 to 14, preferably at 12.75, in order to have maximum amplification factor for a given length of the nozzle with respect to the application. With this range, or pre-stress values, particle size achieved is 46-176 nm (nanometers). Below the range of 1.8 N/sq mm, particle size achieved is 208 nm to 481 nm. Above the range of 0.25 N/sq mm, particle size achieved is >2.75 micrometers or it can fail/damage the piezoelectric crystals.

In at least an embodiment, using the device of this invention, drug flows under gravity through the tubing (112); therefore, there is no need of a pressure injector or any third-party equipment.

The device, of this invention, is configured for creating and delivering aerosol under ultrasound waves. Compared to prior art, this is far more effective and much easier to set-up and deliver. This device does not rely on third party pressure injectors. Since procedures, in respect of palliative care, are repetitive procedures, this device is more conducive to be used, since there is cost reduction, on account of it being an independent, non-third-party-dependent, device.

Preferably, RFID technology is used to prevent the reuse/misuse of the same nozzle, multiple times.

Preferably, the entire device is made of Titanium for the acoustic properties that it lends to the construction of this device.

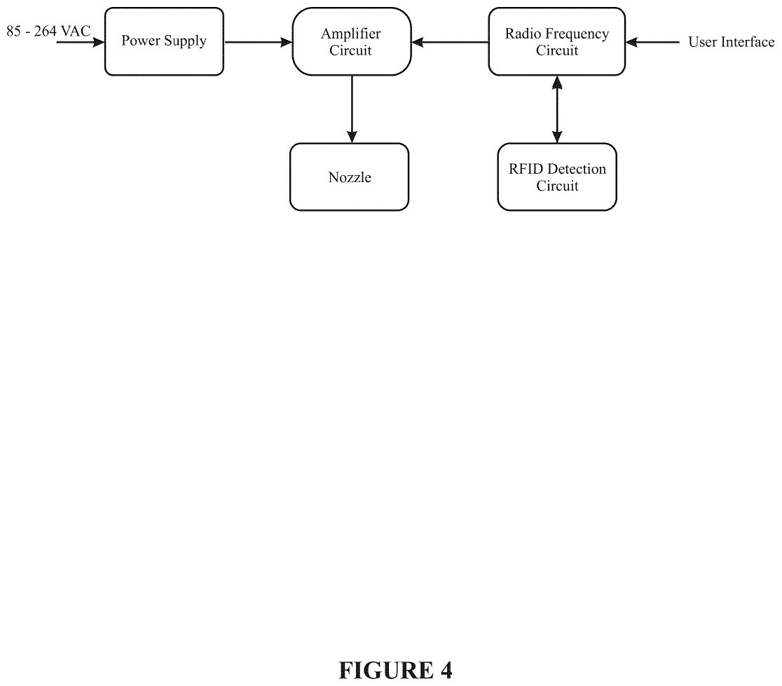

FIG. 4 illustrates a block diagram for the working of the device of this invention.

According to a non-limiting exemplary embodiment, therapy procedures such as PIPAC are to be repeated after every 6 weeks for a palliative patient and for over 6-8 times. Prior art mechanisms require a huge setup; therefore, they cannot be set up in a small-sized hospitals. This prohibits a patient to obtain better care due to availability and affordability issues.

The device, of this invention, however, is independent, and portable; therefore, it is convenient to be used. Thus, this device can be used even in small sizes hospitals; thereby, overcoming availability and affordability issues of the prior art.

It was also observed, by the inventors, that, prior art mechanisms deliver, at the start, few non-aerosolized drops of diluted chemotherapy, as it is, which is not at all desired. In contrast, the device, of the current invention, delivers an aerosolized drug right from the first instance of delivery; there is no lag for delivery of the right particle sized drug.

It was also observed, by the inventors, that, in prior art mechanisms, the average particle size, delivered in an abdomen is in Microns (Micrometers) in size, due to the pressure-based technique of aerosolization. In contrast, average particle size, delivered in the abdomen, using the NAC (Nano Aerosolized Chemotherapy) technique with the device of the invention, is Nanometers in size. Thus ensuring higher depth of penetration in the tissue, better drug concentration in the tissues and better drug distribution on the tissues.

Typically, volume of particles 3 μm is 97.5% and 2.5% of particles 3 μm; using the prior art mechanism, whereas volume of particles 150 nm is 95% and 5% of particles 150 nm but 500 nm using the device of this inventions. All particles are in nanometer range.

It was further observed by the inventors that larger droplets are primarily deposited by impaction and gravitational settling onto a peritoneal surface facing the aerosolizing device of the prior art, leading to poor drug distribution within a peritoneal cavity. In contrast, using the device of the current invention, particles being in nanometre size, ranging from 46 nm to 176 nm; hence can uniformly disperse within the abdominal cavity and can spread even to inaccessible areas; this improves bioavailability and enhances therapy performance.

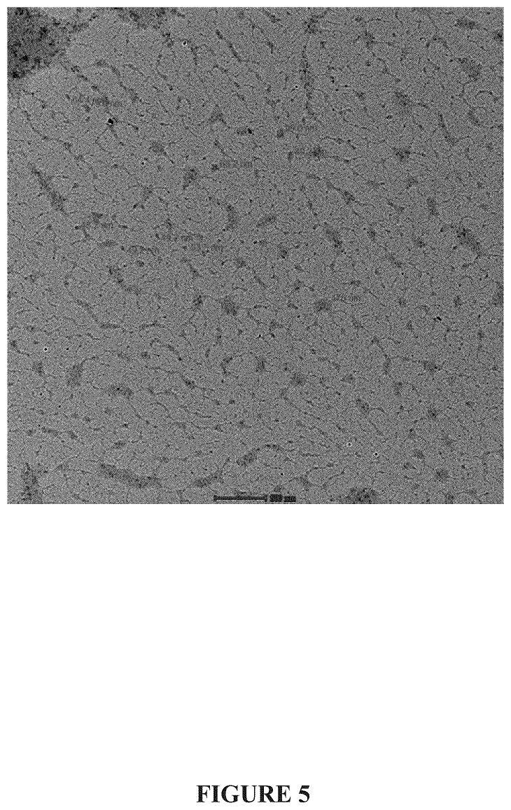

FIG. 5 illustrates that smaller particle size ranging from 46 nm to 176 nm sizes enhances the absorption of drug in the tissue.

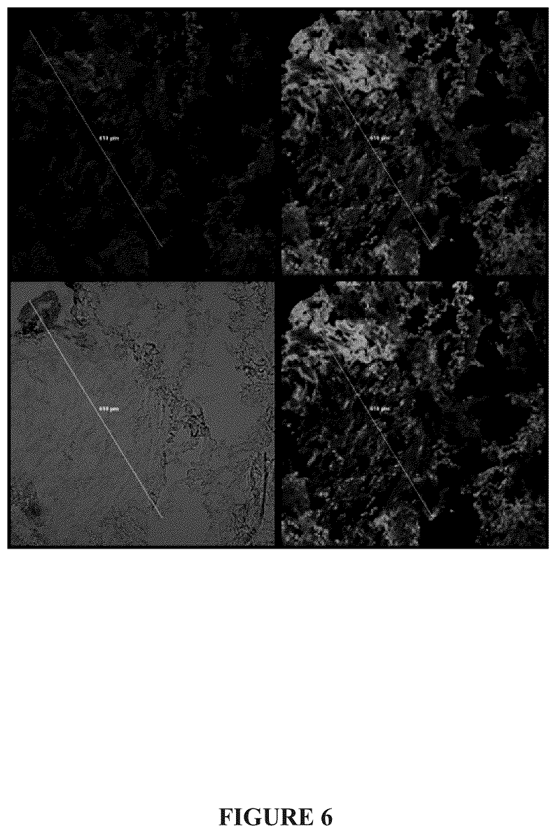

FIG. 6 illustrates increased depth of drug penetration up to 800 μm in the tissue due to smaller drug particle size.

It was further observed that, by using the device of this invention:
uniform drug distribution within the abdominal cavity because of possibility of maneuverability of the nozzle and greater drug concentration in the tissue enhancing the bioavailability of the drug;
no need of a third-party pressure injector for performing procedure, hence can be done in a small sized hospital setup enhancing availability/accessibility to every needy patient; and
tighter particle size bandwidth.

Even though this specification may be written in relation to chemotherapy, it is to be understood that any for of drug can be used in connection with this device.

The TECHNICAL ADVANCEMENT of this invention lies in providing a device which provides ultrasonic atomization of drug for IP therapy; this drug delivery has a much tighter droplet size distribution than prior art's pressurized atomization. Here, atomized drug, under the influence of ultrasound waves, has particle size in nanometers. There is greater depth of penetration, using the device of this invention, and, hence, there is better absorption of drugs; thus, ensuring increased efficacy of the system and better patient outcomes. The flow of drug, using the device of this invention, is gravity fed; this eliminates the need of costly, bulky, and non-portable pressure injector systems typically found in bigger hospitals only and, typically, required by prior art mechanisms. Furthermore, due to narrow bandwidth of nano-particle size, drug distribution, using the device of this invention, is deposited all throughout an abdomen cavity and in a uniform manner.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

What is claimed is:

1. A device for delivery of aerosolized drug in a portion of a body, the device comprising:
a nozzle having a head portion extending distally into an elongate shaft, wherein the head portion includes,
a piezoelectric transducer mounted between a pair of electrically-conductive electrode discs, wherein the piezoelectric transducer is configured to create capillary waves in a liquid film leading to atomization of the drug passing through the nozzle, wherein the piezoelectric transducer has a pre-stress of 1.8 to 3.25 N/sq mm to attain an optimized amplitude of standing waves, and
an aperture at a proximal end of the nozzle configured to connect to tubing configured to carry the drug, wherein the aperture opens to a passage through the elongate shaft, the piezoelectric transducer, and the electrically-conductive electrode discs; and
a body supported by the elongate shaft at a proximal end of the elongate shaft, wherein the elongate shaft has an opening at a distal end, wherein the opening is configured to receive the drug from the tubing and dispense the drug through the opening.

2. The device of claim 1, wherein a ratio of a cross-sectional area of the body to the elongate shaft is 11 to 14 to attain a maximum amplification factor for a given length of the nozzle.

3. The device of claim 1, wherein the piezoelectric transducer is disc-shaped.

4. The device of claim 1, wherein the nozzle has a conical sandblasted surface with roughness configured to reduce cohesive force between liquid atoms resulting in fine atomization.

5. The device of claim 1, wherein the body is with a flat top surface configured to support the nozzle.

6. The device of claim 1, further comprising:
a fastener at a top of the nozzle, wherein the fastener is configured to hold the piezoelectric transducer at the top.

7. The device of claim 1, wherein the body is positioned at a bottom of the nozzle to hold the piezoelectric transducer apart from the bottom.

8. The device of claim 1, further comprising:
the tubing, wherein the tubing is connected to the nozzle and through the aperture by a luer connector.

9. The device of claim 8, wherein the tubing is gravity-fed tubing.

10. The device of claim 1, further comprising:
a cable carrying an electric potential to the piezoelectric transducer.

11. The device of claim 1, wherein the body is cylindrical and extends away from the discs toward the elongate shaft, wherein the elongate shaft has a diameter less than a diameter of the body.

12. The device of claim 1, wherein the elongate shaft includes a conical surface adjacent to the opening, wherein the conical surface tapers such that it is narrowest adjacent to the opening of the elongate shaft.

13. The device of claim 13, wherein the elongate shaft includes a step adjacent to the conical surface opposite the opening.

14. The device of claim 1, further comprising:
a controller configured to control frequency of vibration of the piezoelectric transducer which atomizes drug from its liquid form into a nano-particle mist before being dispensed through the opening.

15. The device of claim 13, the frequency is 20-80 KHz.

16. The device of claim 1, wherein the elongate shaft and body are fabricated entirely of titanium metal and/or titanium alloy.

17. A method of using the device of claim 1, comprising:
flowing a drug under the force of gravity through the tubing;
atomizing the drug in the tubing by generating capillary waves with the piezoelectric transducer; and
ejecting the atomized drug from the opening of the elongate shaft.

18. The method of claim 16, wherein the atomizing uses capillary waves having frequency 20-80 KHz.
